(19) United States
(12) Patent Application Publication
(10) Pub. No.: US 2022/0214305 A1
(43) Pub. Date: Jul. 7, 2022

(54) METHOD AND DEVICE FOR DETERMINING AN INTERNAL RESISTANCE OF A SENSOR ELEMENT

(71) Applicant: Robert Bosch GmbH, Stuttgart (DE)

(72) Inventors: Anne-Katrin Mittasch, Reutlingen (DE); Stefan Haller, Filderstadt (DE)

(21) Appl. No.: 17/557,724

(22) Filed: Dec. 21, 2021

(30) Foreign Application Priority Data
Jan. 4, 2021 (DE) 10 2021 200 004.5

(51) Int. Cl. G01N 27/416; G01N 27/12; G01N 33/00; G01N 27/407

(52) U.S. Cl. CPC G01N 27/4163; G01N 27/407; G01N 33/0027; G01N 27/12

(57) ABSTRACT

A method for ascertaining an internal resistance of a sensor element. The method includes: ascertaining a reference voltage between a first electrode and a second electrode; impressing of a first current pulse having a first current by a pulse-generating unit at a first time; ascertaining at least two voltage values at two different times after an elapsing of a first settling time after the first time; ending the first current pulse and impressing an opposite second current pulse having a second current at a second time, ending the second current pulse at a third time; ascertaining a linear equation as a function of the at least two voltage values and the times; extrapolating a voltage value at the first time using the linear equation; ascertaining an internal resistance of the sensor element as a function of the extrapolated voltage value, the reference voltage, and the first current.

### CROSS REFERENCE

The present application claims the benefit under 35 U.S.C. § 119 of German Patent Application No. DE 102021200004.5 filed on Jan. 4, 2021, which is expressly incorporated herein by reference in its entirety.

### FIELD

The present invention relates to a method for determining an internal resistance of a sensor element, and to a computer program.

### BACKGROUND INFORMATION

PCT Patent Application No. WO 2016/173814 A1 describes a method for determining an internal resistance of a sensor element (110) for acquiring a portion of a gas component from a gas mixture in a measurement gas space, which is intended to enable a determination that is as accurate as possible of the internal resistance of the sensor element (110). The sensor element (110) has at least one cell (114), the cell (114) having at least one first electrode (116), at least one second electrode (118), and at least one solid electrolyte (120) that connects the first electrode (116) and the second electrode (118), an electrical voltage (124) being present between the first electrode (116) and the second electrode (118).

### SUMMARY

The present invention relates to a method for determining an internal resistance of a sensor element. In addition, the present invention relates to a computer program that is set up to carry out one of the methods.

In a first aspect of the present invention, a method is provided for ascertaining an internal resistance of a sensor element for acquiring a gas component from a gas mixture in a measurement gas space, the sensor element having at least one cell, the cell having at least one first electrode, at least one second electrode, connecting solid electrolytes, an electrical voltage being measurable between the first and the second electrode, the method including the following steps:
ascertaining a reference voltage between the first electrode and the second electrode,
impressing a first current pulse with a first current using a pulse-generating unit at a first time,
   the first current pulse bringing about a charge shift in the sensor element,
   the occurrence of the charge shift causing an increase in the electrical voltage between the first electrode and the second electrode,
ascertaining at least two voltage values at two different times, after an elapsing of a first specifiable settling time after the first time, between the first electrode and the second electrode,
ending the first current pulse and impressing an opposite second current pulse with a second current at a second time, the opposite second current pulse causing a depolarization between the first electrode and the second electrode, as well as a charge shift,
ending the second current pulse at a third time,
ascertaining a linear equation as a function of the at least two voltage values and times,
extrapolation of a voltage value at the first time using the linear equation,
ascertaining an internal resistance of the sensor element as a function of the extrapolated voltage value and of the reference voltage and of the first current of the first current pulse.

The method in accordance with the present invention has the particular advantage that the polarization-dependent portion of the voltage increase is assumed as linear. Consequently, from the time curve, assumed as linear, of the voltage applied to the cell during the charge shift the polarization-dependent portion of the increase can be extrapolated in linear fashion. The value ascertained in this way for the polarization-dependent portion of the increase of the electrical voltage in the cell can, as described above, consequently be used for the more accurate determination of the value of the internal resistance of the sensor element.

The value ascertained by this method for the polarization-dependent portion of the increase of the electrical voltage in the cell can, as described above, consequently be used for the more precise ascertaining of the value for the internal resistance of the sensor element.

The method according to the present invention is in addition advantageous because a linear extrapolation is easy to program, and is implemented in a manner that saves resources for calculation for the control device.

Consequently, by the more precise ascertaining of the internal resistance of the sensor element, a more precise temperature can be ascertained for the sensor element, so that a more accurate thermal management for the sensor element can be carried out.

A further advantage is that the first and the second current pulse can be kept short, because only two measurement values have to be carried out during the voltage curve, assumed as linear. Thus, the probe can be reused more quickly for measurement of the oxygen concentration of the exhaust gas.

In a second variant of the present invention, a method is proposed for ascertaining an internal resistance of a sensor element for acquiring a gas component from a gas mixture in a measurement gas space, the sensor element having at least one cell, the cell including at least one first electrode, at least one second electrode, and connecting solid electrolytes, such that an electrical voltage is measurable between the first and the second electrode, the method including the following steps:
ascertaining a reference voltage between the first electrode and the second electrode,
impressing a first current pulse with the first current using a pulse generating unit at a first time,
   the first current pulse bringing about a charge shift in the sensor element,
   the occurrence of the charge shift causing an increase in the electrical voltage between the first electrode and the second electrode,
ascertaining at least one voltage value between the first electrode and the second electrode at a time after an elapsing of a first settling time after the first time,
ending the first current pulse and impressing an opposite second current pulse with a second current at a second time, the opposite second current pulse causing a depolarization between the first electrode and the second electrode as well as a charge shift,
ascertaining at least two voltage values between the first electrode and the second electrode at two different times after an elapsing of a second specifiable settling time after the second time,
ending the second current pulse at a third time,
ascertaining a second slope of a straight line through the at least two voltage values at two different times,
ascertaining a voltage value at the first time as a function of the first and of the second current and of the ascertained second slope,
ascertaining the internal resistance of the sensor element as a function of the ascertained voltage value and of the reference voltage and of the first current of the first current pulse.

The value ascertained by this example method for the polarization-dependent portion of the increase of the electrical voltage in the cell can, as described above, consequently be used for the more precise ascertaining of the value for the internal resistance of the sensor element.

The method in accordance with the present invention is further advantageous because the calculation in the control device, through the voltage curve assumed as linear, is easy to program and can be realized in a manner that saves resources.

Consequently, using the more precise ascertaining of the internal resistance of the sensor element, a more precise temperature for the sensor element can be ascertained, so that a more accurate thermal management for the sensor element can be carried out.

Through the inclusion of the polarization portion, assumed as linear, of the voltage during the opposite current pulse, a further increase of the precision for ascertaining the internal resistance can be achieved.

In a third variant of the present invention, a method is provided for ascertaining an internal resistance of a sensor element for acquiring a gas component from a gas mixture in a measurement gas space, the sensor element having at least one cell, the cell including at least one first electrode, at least one second electrode, and a connecting solid electrolyte, an electrical voltage being measurable between the first and the second electrode, the method including the following steps:
ascertaining at least two voltage values during a first time duration, starting at the time and ending at a time, preferably having a time duration of 10 ms, between the first electrode and the second electrode,
ascertaining a third slope as a function of the ascertained at least two voltage values during a first time duration,
impressing a first current pulse with a first current using a pulse-generating unit at a first time,
   the first current pulse causing a charge shift in the sensor element,
   the occurrence of the charge shift causing an increase in the electrical voltage between the first electrode and the second electrode,
ascertaining at least one voltage value between the first electrode and the second electrode at a time after an elapsing of a first settling time after the first time,
ending the first current pulse and impressing an opposite second current pulse having a second current at a second time, the opposite second current pulse causing a depolarization between the first electrode and the second electrode, as well as a charge shift,
ascertaining at least two voltage values between the first electrode and the second electrode at two different times after elapsing of a second specifiable settling time after the second time,
ending the second current pulse at a third time,
ascertaining a second slope of a straight line through the at least two voltage values at two different times,
ascertaining a voltage value at the first time as a function of the first and of the second current, of the ascertained second slope, and of a corrected slope,
ascertaining the internal resistance of the sensor element as a function of the ascertained voltage value and of the reference voltage and of the first current of the first current pulse.

The value ascertained by this example method for the polarization-dependent portion of the increase of the electrical voltage in the cell can, as described above, consequently be used for the more precise ascertaining of the value for the internal resistance of the sensor element.

The method in accordance with the present invention is further advantageous because the calculation in the control device, using the voltage curve assumed as linear, is easy to program and can be realized in a resource-saving manner.

Consequently, using the more precise ascertaining for the internal resistance of the sensor element, a more precise temperature can be ascertained for the sensor element, so that a more accurate thermal management for the sensor element can be carried out.

Through the inclusion of the polarization portion, assumed as linear, of the voltage during the opposite current pulse, a further increase of the precision for ascertaining the internal resistance can be achieved. The ascertaining and use of the third slope for ascertaining the internal resistance is done under the assumption that modifications of the oxygen concentration in the exhaust gas during a measurement cause a change in the voltage. This effect can thus easily influence the ascertaining of the internal resistance.

In addition, the specifiable first settling time and the second specifiable second settling time can be determined as a function of component properties of a low-pass filter.

In addition, the sensor element that is connected via a low-pass filter can, the low-pass filter being connected to a control device, the low-pass filter having associated time constants, a first time for the ascertaining of a first value for the increase of the electrical voltage being selected such that the first time corresponds at least to three times, preferably at least five times, the time constant of the low-pass filter.

In further aspects, the present invention relates to a device, in particular to a control device and to a computer program, that are set up, in particular programmed, to carry out one of the methods. In a still further aspect, the present invention relates to a machine-readable storage medium on which the computer program is stored.

### BRIEF DESCRIPTION OF THE DRAWINGS

In the following, the present invention is described in more detail with reference to the figures, and on the basis of exemplary embodiments.

FIG. 1 shows a schematic representation of an electrical wiring of a sensor element.

FIG. 2 shows a schematic representation of the time curve of the electrical voltage between the first electrode and the second electrode of the sensor element.

FIG. 3 shows a first example of a sequence of an exemplary embodiment of the method of the present invention, via a flow diagram.

FIG. 4 shows a second example of a sequence of an exemplary embodiment of the method of the present invention, via a flow diagram.

FIG. 5 shows a third example of a sequence of an exemplary embodiment of the method of the present invention, via a flow diagram.

### DETAILED DESCRIPTION OF EXAMPLE EMBODIMENTS

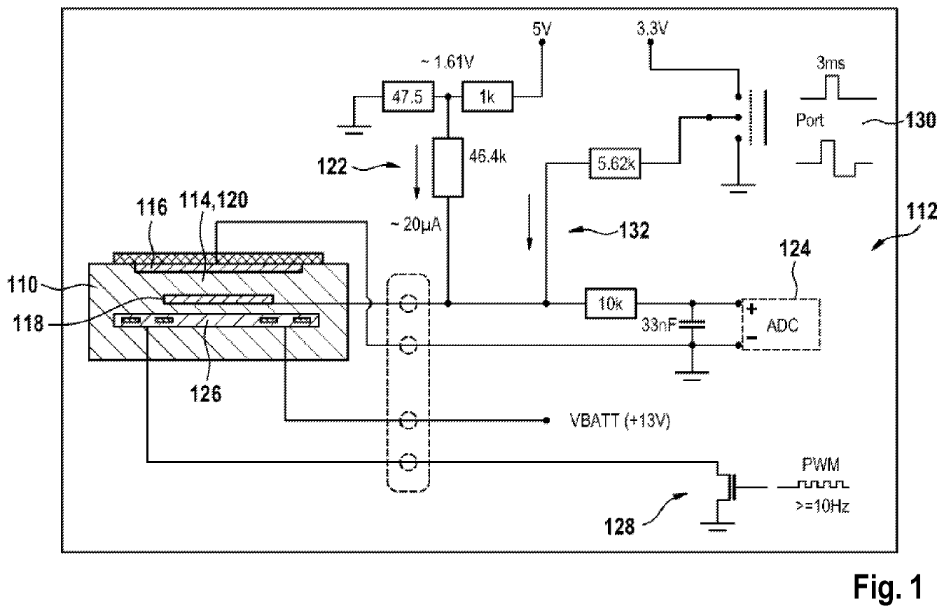

FIG. 1 schematically shows a sensor element 110 for acquiring a portion of a gas component from a gas mixture in a measurement gas space, as well as the associated electrical wiring 112. Sensor element 110, shown here as an example, has a cell 114 that has a first electrode 116, a second electrode 118, and a solid electrolyte 120 that connects the first electrode 116 and the second electrode 118.

The two electrodes are preferably made of zirconium dioxide. In a preferred embodiment, the first electrode 116 is connected with the measurement gas space via a porous protective layer, while the second electrode 116 is situated in an electrode hollow space that is preferably charged with gas from the measurement gas space via at least one diffusion barrier. As described above, a fixed voltage is applied between the first electrode and the second electrode of the cell. As soon as an oxygen concentration in the electrode hollow space is close to 0, a Nernst potential increases strongly, and partly compensates the applied voltage. In this way, a constant oxygen concentration can be set in the electrode hollow space with a good degree of precision. Sensor element 110, shown here as an example, has a cell 114 that has a first electrode 116, a second electrode 118, and a solid electrode 120 connecting the first electrode 116 and the second electrode 118. By applying a current 122 to cell 114, an electrical voltage 124 between first electrode 116 and second electrode 118 can be determined using a suitable voltage detection device. Sensor element 110 shown here additionally has a heating element 126 that can be operated using an associated heat control unit 128 in such a way that the temperature of sensor element 110 can thereby be set.

Using a pulse-generating unit 132, a current pulse 130 can be applied to sensor element 110, or to cell 114. The charging of sensor element 110 with current pulse 130 causes an occurrence of a charge shift in sensor element 110 that is expressed as a measurable increase in the electrical voltage 124 in cell 114 between first electrode 116 and second electrode 118.

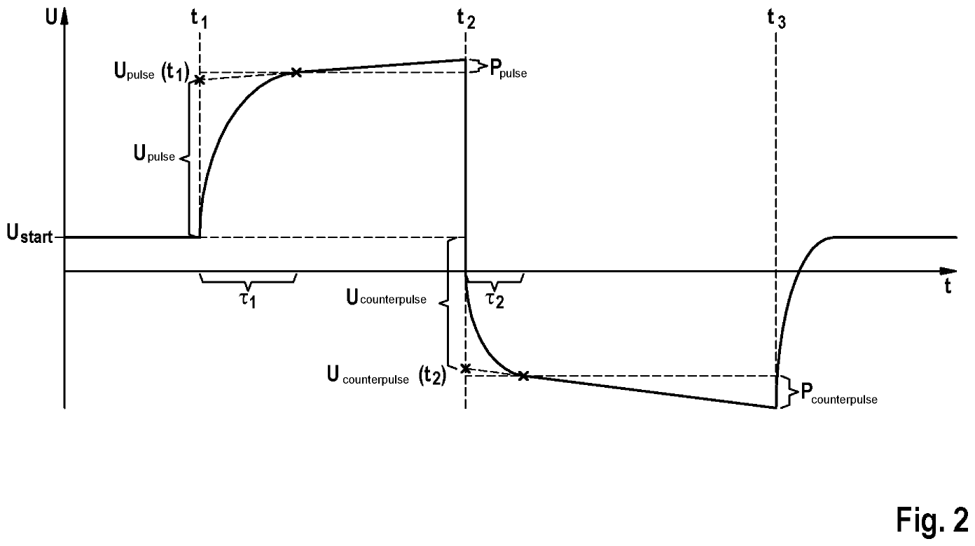

FIG. 2 shows a time curve of the electrical voltage U of cell 114. Initially, electrical voltage U of cell 114 is at a voltage value Uₛₜₐᵣₜ, or reference voltage Uₛₜₐᵣₜ. At a first time t₁, a pulse-generating unit 132 impresses a first current pulse Iₚᵤₗₛₑ having a current I₁, or a current strength I₁, onto cell 114 until a second time t₂. During this time span Δt₁₂, voltage U has both an ohmic portion Uₚᵤₗₛₑ and a polarization-dependent portion Pₚᵤₗₛₑ. The polarization-dependent voltage curve can be regarded as approximately linear after about a first settling time τ₁ after the impressing of the first current pulse Iₚᵤₗₛₑ, i.e. starting from the time t₁+τ₁. At a second time t₂>t₁+τ₁, first current pulse Iₚᵤₗₛₑ ends, and an opposite second current pulse I_{counterpulse}, having a current I₂, or a current strength I₂, is carried out by pulse-generating unit 132 until third time t₃. Here, “opposite” means that first current pulse Iₚᵤₗₛₑ has a different sign from second current pulse I_{counterpulse}, and the current strengths I₂ and I₂ can differ in their magnitude. The opposite second current pulse I_{counterpulse} provides a depolarization of cell 114, and shows an opposite symmetrical curve for voltage U. That is, here as well an ohmic portion U_{counterpulse} and a polarization-dependent portion P_{counterpulse} can be recognized.

The polarization-dependent voltage curve can be regarded as linear after approximately a second settling time τ₂ after the impressing of the second current pulse I_{counterpulse}, i.e. starting from the time t₂+τ₂. At third time t₃>t₂+τ₂, opposite second current pulse I_{counterpulse} ends and the voltage again assumes its initial voltage Uₛₜₐᵣₜ.

Using, for example, a linear approximation in the time intervals [t₁+τ₁; t₂] and [t₂+τ₂; t₃], the two polarization-adjusted voltage values Uₚᵤₗₛₑ(t₁) U_{counterpulse}(t₂) can be ascertained by extrapolation at first time t₁ and at second time t₂. Subsequently, through simple subtraction of the polarization-adjusted voltage values Uₚᵤₗₛₑ(t₁) U_{counterpulse}(t₂) and the voltage value Uₛₜₐᵣₜ ascertained initially, the internal resistance R of cell 114 can be ascertained.

The internal resistance R of cell 114 results as:

$$R = \frac{{U}_{pulse ( {t}_{1} )} - {U}_{start}}{{I}_{1}} \\ R = \frac{{U}_{counterpulse ( {t}_{2} )} - {U}_{start}}{{I}_{2}}$$

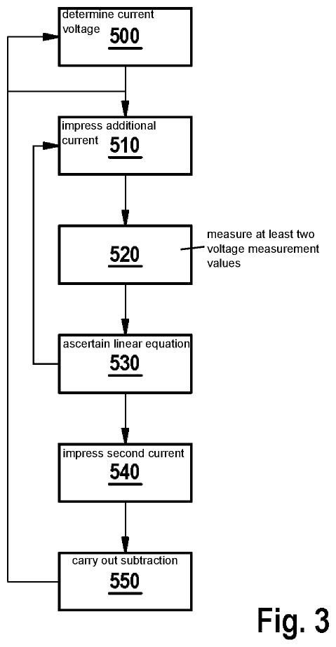

FIG. 3 shows the exemplary sequence of the method for determining an internal resistance R of a sensor element 110.

In a first step 500, using the measurement system shown in FIG. 1, the current voltage Uₛₜₐᵣₜ of sensor element 110, in particular of cell 114, without additional current loading is determined. The ascertained voltage value Uₛₜₐᵣₜ of sensor element 110 is here received by control device 100 and is later stored.

Alternatively or in addition, a plurality of measurements can be carried out for a specifiable time duration and a subsequent averaging over the ascertained voltage values.

Subsequently, the method is continued in a step 510.

In a step 510, using pulse-generating unit 132 an additional current I₁ is impressed onto sensor element 110, in particular onto cell 114. The charging of sensor element 110 with first current pulse Iₚᵤₗₛₑ brings about a charge shift in cell 114, which causes an increase in the voltage in cell 114 between first electrode 116 and second electrode 118.

The impressing of the first current pulse Iₚᵤₗₛₑ takes place at a first time t₁ and ends at a second time t₂; that is, first current pulse Iₚᵤₗₛₑ has a specifiable time duration Δt₁₂=t₂ t₁.

Specifiable time duration Δt₁₂ is preferably selected as a function of the component of the low-pass filter (ADC). This can be carried out for example in an application phase.

Subsequently, the method is continued in a step 520.

In a step 520, at least two voltage measurement values U₁, U₂ are measured at different times t_{U1} and t_{U2}. The measurement of the at least two voltage measurement values U₁, U₂ is here first carried out when a first specifiable settling time τ₁<Δt₁₂, which is preferably selected as a function of the low-pass filter (ADC) that is used, has elapsed. This can be carried out for example in an application phase. The measurement is first carried out at the beginning of the first current pulse Iₚᵤₗₛₑ, started in step 510, and is carried out after the elapsing of the first settling time τ₁, i.e. after a time duration tₘₑₐₛ=t₁+τ₁, so that t_{U1}≥tₘₑₐₛ, t_{U2}>t_{U1}. The at least two measurement values U₁, U₂, the times t_{U1}, t_{U2} and the current I₁ of the first current pulse Iₚᵤₗₛₑ are acquired and stored by control device 100 for this purpose. Subsequently, the method is continued in step 530.

In step 530, as a function of the at least two voltage measurement values U₁, U₂ and the associated times t_{U1}, t_{U2}, a linear equation G₁ is ascertained, and subsequently, using linear extrapolation, the voltage value Uₚᵤₗₛₑ(t₁) at first time t₁ is ascertained and stored by control device 100. Subsequently, the method is continued in step 540. In an alternative specific embodiment, a plurality of measurements i=1, 2, . . . , n, with n∈ [symbol] , as in step 520, can be carried out with different current pulses. Subsequently, an averaging of the back-calculated voltage values Ū(t₁)=Σᵢ₌₁ⁿUᵢ(t₁) can be carried out. Subsequently, the method can be continued in step 540, using the averaged voltage value.

In a step 540, at second time t₂ pulse-generating unit 132 is used to impress a specifiable second current I_{counterpulse}, in the direction opposite to first current pulse Iₚᵤₗₛₑ, onto sensor element 110. In this way, a depolarization of sensor element 110, or of cell 114, takes place. The impressing of second current pulse I_{counterpulse} takes place at a second time t₂ and ends with a third time t₃; that is, second current pulse I_{counterpulse} has a specifiable time duration Δt₂₃=t₃−t₂. With the ending of the second current pulse, i.e. at a third time t₃, the method continues in step 550.

In a step 550, using control device 100 a subtraction is subsequently carried out between the voltage value Uₚᵤₗₛₑ(t₁) extrapolated in step 530 at first time t₁ and the voltage value Uₛₜₐᵣₜ ascertained in step 500.

Subsequently, from the ascertained voltage value Uₚᵤₗₛₑ=Uₚᵤₗₛₑ(t₁)−Uₛₜₐᵣₜ a corrected internal resistance R is ascertained for sensor element 100, or cell 114:

$$R = \frac{{U}_{pulse}}{{I}_{1}}$$

where Uₚᵤₗₛₑ is the difference between the extrapolated voltage value Uₚᵤₗₛₑ(t₁), the current I₁, and the ascertained voltage value Uₛₜₐᵣₜ.

Subsequently, the method can be started from the beginning, in step 500, or can be ended.

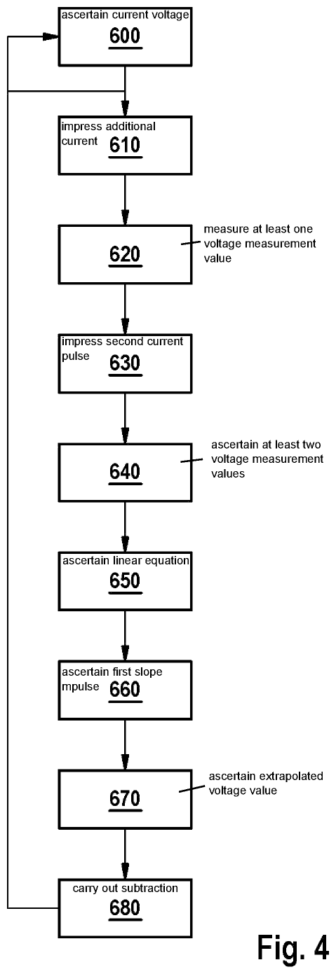

FIG. 4 shows an alternative sequence for the method for determining an internal resistance of a sensor element 110, in particular cell 114.

In a first step 600, using the measurement system shown in FIG. 1 the current voltage Uₛₜₐᵣₜ of sensor element 110, in particular of cell 114, without additional current loading is ascertained. The ascertained voltage value Uₛₜₐᵣₜ of sensor element 110 is here received by control device 100 and is later stored.

Alternatively or in addition, it is also possible to carry out a plurality of measurements for a specifiable time duration and a subsequent averaging over the ascertained voltage values.

Subsequently, the method is continued in a step 610.

In a step 610, pulse-generating unit 132 is used to impress an additional current I₁ onto sensor element 110, in particular onto cell 114. The charging of sensor element 110 with the first current pulse Iₚᵤₗₛₑ causes a charge shift in cell 114 that results in an increase of the voltage in cell 114 between first electrode 116 and second electrode 118.

The impressing of first current pulse Iₚᵤₗₛₑ takes place at a first time t₁ and ends at a second time t₂; i.e. first current pulse Iₚᵤₗₛₑ has a specifiable time duration Δt₁₂=t₂ t₁.

The specifiable time duration Δt₁₂ is preferably selected as a function of the component of the low-pass filter (ADC). This can be carried out for example in an application phase.

Subsequently, the method is continued in a step 620.

In a step 620, at least one voltage measurement value U₁ is measured at time t_{U1}. The measurement of the at least one voltage measurement value U₁ is here first carried out when a first specifiable settling time τ₁, which is preferably selected as a function of the low-pass filter (ADC) that is used, has elapsed. This can be carried out for example in an application phase.

The measurement is first carried out with the beginning of first current pulse Iₚᵤₗₛₑ, started in step 610, and after the elapsing of the first settling time τ₁<Δt₁₂, i.e. after the time tₘₑₐₛ=t₁+τ₁, so that t_{U1}≥tₘₑₐₛ. The at least one measurement value U₁, the at least one time t_{U1}, and current I₁ of the first current pulse Iₚᵤₗₛₑ are acquired and stored by control device 100 for this purpose. It is assumed that the rise of voltage U starting at time t₁+τ₁ is caused approximately solely by polarization effects.

Subsequently, the method is continued in step 630.

In a step 630, at second time t₂ pulse-generating unit 132 is used to impress a specifiable second current pulse I_{counterpulse}, in the opposite direction to first current pulse Iₚᵤₗₛₑ, onto sensor element 110. As a result, a depolarization of sensor element 110, or of cell 114, takes place. The impressing of the second current pulse I_{counterpulse} takes place at a second time t₂, and ends at a third time t₃, i.e. second current pulse I_{counterpulse} has a specifiable time duration Δt₂₃=t₃−t₂. It is assumed that the rise of voltage U starting at time t₂+τ₂ is caused approximately solely by polarization effects. Here, τ₂<Δt₂₃ is a specifiable second settling time.

The specifiable second settling time τ₂ and the specifiable time duration Δt₂₃>τ₂ are selected as a function of the installed low-pass filter (ADC). This can be carried out for example in an application phase.

Subsequently, the method is continued in step 640.

In step 640, after the elapsing of a second settling time τ₂ at least two voltage measurement values W₁, W₂ are ascertained and stored by control device 100 at different times t_{W1} and t_{W2}. The measurement of the at least two voltage measurement values W₁, W₂ is first carried out after the beginning of the second current pulse I_{counterpulse}, started in step 630, and after the elapsing of the second settling time τ₂, i.e. at the earliest starting at a time tₘₑₐₛ₂=t₂+τ₂, so that t_{W1}≥tₘₑₐₛ₂, t_{W2}>t_{W1}. The at least two measurement values W₁, W₂, the corresponding at least two times t_{W1}, t_{W2}, and the current I₂ of second current pulse I_{counterpulse} are acquired and stored by control device 100 for this purpose.

Subsequently, the method is continued in step 650.

In a step 650, a linear equation G₂ having a second slope m_{counterpulse} is subsequently ascertained from the two voltage values W₁, W₂ ascertained in step 640 and the associated times t_{W1}, t_{W2}. Here it is assumed that the curve of voltage U can be linearly approximated starting from time t₂+τ₂.

Subsequently, the method is continued in step 660.

In a step 660, as a function of the ascertained second slope m_{counterpulse} of the linear curve during the opposite second current pulse I_{counterpulse} and the ascertained currents I₁ of the first current pulse Iₚᵤₗₛₑ and the [ . . . ] I₂ of the second current pulse I_{counterpulse}, the first slope mₚᵤₗₛₑ of the polarization portion, assumed as linear, during the first current pulse Iₚᵤₗₛₑ is ascertained as follows:

$${m}_{pulse} = {m}_{counterpulse} \cdot ( \frac{{I}_{1}}{{I}_{2}} )$$

with m_{counterpulse} of the second slope of the voltage curve U, assumed as linear, or of straight lines G₂ during second current pulse I_{counterpulse}, current I₁ during the first current pulse Iₚᵤₗₛₑ and second current I₂ during second current pulse I_{counterpulse}.

Subsequently, the method is continued in step 670.

In a step 670, using the first voltage value U₁ ascertained in step 620 and its time t_{U1}, the first slope mₚᵤₗₛₑ, ascertained in step 660, and first time t₁, the extrapolated voltage value Uₚᵤₗₛₑ (t₁) is ascertained.

Uₚᵤₗₛₑ(t₁)=U₁−(mₚᵤₗₛₑ*(t_{U1}−t₁))

Subsequently, the method is continued in step 680.

In a step 680, using control device 100 a subtraction is subsequently carried out between the extrapolated voltage value Uₚᵤₗₛₑ(t₁) and the voltage value Uₛₜₐᵣₜ ascertained in step 600.

Subsequently, from the ascertained voltage value Uₚᵤₗₛₑ=Uₚᵤₗₛₑ(t₁)−Uₛₜₐᵣₜ a corrected internal resistance R is ascertained for sensor element 110, or for cell 114:

$$R = \frac{{U}_{pulse}}{{I}_{1}} ,$$

with Uₚᵤₗₛₑ the difference between the extrapolated voltage value Uₚᵤₗₛₑ(t₁) and the voltage value Uₛₜₐᵣₜ ascertained in step 600, the current I₁, and the ascertained voltage value Uₛₜₐᵣₜ.

Subsequently, the method can be started from the beginning in step 600, or can be ended.

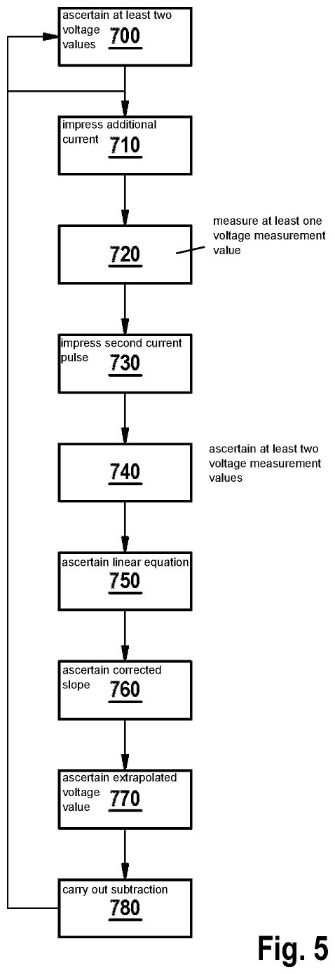

FIG. 5 shows a third alternative sequence of the method for determining an internal resistance of a sensor element 110, in particular of cell 114.

In a first step 700, using the measurement system shown in FIG. 1 at least two specifiable voltage values U_{start,i} of sensor element 110, in particular of cell 114, without additional current loading are ascertained, with i=1, 2, . . . , n, n∈ [symbol] . This takes place within a time duration Δₛₜₐᵣₜ starting at a time t₀ and ending at a time t₁. The time duration Δₛₜₐᵣₜ can here be for example several milliseconds, preferably 10 ms.

Subsequently, as a function of the ascertained voltage values U_{start,i} and the associated times t_{start,i} a linear equation G₃ having a third slope mₛₜₐᵣₜ is ascertained.

Subsequently, the method is continued in a step 710.

In a step 710, pulse-generating unit 132 is used to impress an additional current I₁ onto sensor element 110, in particular onto cell 114. The charging of sensor element 110 with first current pulse Iₚᵤₗₛₑ causes a charge shift in cell 114, which causes an increase of the voltage in cell 114 between first electrode 116 and second electrode 118.

The impressing of the first current pulse Iₚᵤₗₛₑ takes place at a first time t₁ and ends at a second time t₂; i.e., the first current pulse Iₚᵤₗₛₑ has a specifiable time duration Δt₁₂=t₂−t₁.

The specifiable time duration Δt₁₂ is preferably selected as a function of the component of the low-pass filter (ADC). This can be carried out for example in an application phase.

Subsequently, the method is continued in a step 720.

In a step 720, at least one voltage measurement value U₁ is measured at time t_{U1}. The measurement of the at least one voltage measurement value U₁ is first carried out when a first specifiable settling time τ₁<Δt₁₂, preferably selected as a function of the low-pass filter (ADC) that is used, has elapsed. This can be carried out for example in an application phase.

The measurement is first carried out with the beginning of the first current pulse Iₚᵤₗₛₑ, started in step 710, and after elapsing of the first settling time τ₁, i.e. after the time tₘₑₐₛ=t₁+τ₁. The at least one measurement value U₁, the time t_{U1}, where t_{U1}−tₘₑₐₛ, and the current I₁ of the first current pulse Iₚᵤₗₛₑ are acquired and stored by control device 100 for this purpose. It is assumed that the rise starting from time t₁+τ₁ of the voltage U is caused approximately solely by polarization effects.

Subsequently, the method is continued in step 730.

In a step 730, at second time t₂ pulse-generating unit 132 is used to impress a specifiable second current pulse I_{counterpulse}, in the direction opposite to first current pulse Iₚᵤₗₛₑ, onto sensor element 110. As a result, a depolarization of sensor element 110, or of cell 114, takes place. The impression of the second current pulse I_{counterpulse} takes place at a second time t₂ and ends at a third time t₃; i.e., second current pulse I_{counterpulse} has a specifiable time duration Δt₂₃=t₃−t₂. It is assumed that the rise of voltage U starting at time t₂+τ₂ is caused approximately solely by polarization effects. Here τ₂<Δt₂₃ is a specifiable second settling time τ₂.

The specifiable time duration Δt₂₃ and the specifiable settling time τ₂ are preferably selected as a function of the installed low-pass filter (ADC). This can be carried out for example in an application phase.

Subsequently, the method is continued in step 740.

In step 740, after the elapsing of a second settling time τ₂ at least two voltage measurement values W₁,W₂ are ascertained and stored by control device 100 at different times t_{W1} and τ_{W2}. The measurement of the at least two voltage measurement values W₁,W₂ is first carried out when the specifiable second settling time τ₂ has elapsed. The measurement is first carried out with the beginning of the second current pulse I_{counterpulse} started in step 630, and is carried out after the elapsing of the second settling time τ₂, i.e. not until after a time tₘₑₐₛ₂=t₂+τ₂, so that t_{W1}≥tₘₑₐₛ₂, t_{W2}≥t_{W1}. The at least two measurement values W₁,W₂, the times t_{W1},t_{W2}, and the current I₂ of the second current pulse I_{counterpulse} are acquired and stored by control device 100 for this purpose.

Subsequently, the method is continued in step 750.

In a step 750, a linear equation G₂ having a second slope m_{counterpulse} is subsequently ascertained from the second voltage values W₁,W₂, ascertained in step 740, and the associated times t_{W1},t_{W2}. Here it is assumed that the curve of voltage U starting from time t₂+τ₂ can be linearly approximated.

Subsequently, the method is continued in step 760.

In a step 760, as a function of the ascertained second slope m_{counterpulse} of the linear curve during the opposite second current pulse I_{counterpulse}, the third slope mₛₜₐᵣₜ and the impressed first current I₁ and the impressed second current I₂ during the first current pulse Iₚᵤₗₛₑ and the second current pulse I_{counterpulse}, the corrected slope m_{pulse,corr} of the polarization portion, assumed as linear, during the first current pulse Iₚᵤₗₛₑ is ascertained as follows:

$${m}_{pulse , corr} = ( {m}_{counterpulse} - {m}_{start} ) \cdot ( \frac{{I}_{1}}{{I}_{2}} ) + {m}_{start}$$

Subsequently, the method is continued in step 770.

In a step 770, the extrapolated voltage value Uₚᵤₗₛₑ(t₁) is ascertained using the first voltage value U₁ ascertained in step 720 and its time t_{U1}, the ascertained corrected slope m_{pulse,corr} and the first time t₁ of the extrapolated voltage value Uₚᵤₗₛₑ(t₁).

Subsequently, the method is continued in step 780.

In a step 780, control device 100 carries out a subtraction between the extrapolated voltage value Uₚᵤₗₛₑ(t₁) and the voltage value Uₛₜₐᵣₜ ascertained in step 700.

Subsequently, from the ascertained voltage value Uₚᵤₗₛₑ=Uₚᵤₗₛₑ(t₁)−Uₛₜₐᵣₜ a corrected internal resistance R is ascertained for sensor element 110, or for cell 114:

$$R = \frac{{U}_{pulse}}{{I}_{1}}$$

where Uₚᵤₗₛₑ is the difference between the extrapolated voltage value Uₚᵤₗₛₑ(t₁), the current I₁, and the ascertained voltage value Uₛₜₐᵣₜ.

Subsequently, the method can be started from the beginning in step 700, or can be ended.

What is claimed is:

1. A method for ascertaining an internal resistance of a sensor element for acquiring a gas component from a gas mixture in a measurement gas space, the sensor element having at least one cell, the cell including at least one first electrode, at least one second electrode, and connecting solid electrolytes, an electrical voltage being capable of being measured between the first electrode and the second electrode, the method comprising the following steps:
ascertaining a reference voltage between the first electrode and the second electrode;
impressing a first current pulse having a first current by a pulse-generating unit at a first time, the first current pulse causing a charge shift in the sensor element, occurrence of the charge shift causing an increase in the electrical voltage between the first electrode and the second electrode;
ascertaining between the first electrode and the second electrode at least two voltage values at two different times after an elapsing of a first specifiable settling time after the first time;
ending the first current pulse and impressing an opposite second current pulse having a second current at a second time, the opposite second current pulse causing a depolarization between the first electrode and the second electrode, and a charge shift;
ending the second current pulse at a third time;
ascertaining a linear equation as a function of the at least two voltage values and the two different times;
extrapolating a voltage value at the first time using the linear equation; and
ascertaining the internal resistance of the sensor element as a function of the extrapolated voltage value and the reference voltage and the first current of the first current pulse.

2. The method as recited in claim 1, wherein the specifiable first settling time is determined as a function of a component property of a low-pass filter.

3. The method as recited in claim 1, wherein the sensor element is connected via a low-pass filter, the low-pass filter being connected to a control device, the low-pass filter having an associated time constant, the first time for the impressing of the first current pulse for increasing of the electrical voltage being selected such that the first time corresponds to at least three times the time constant of the low-pass filter.

4. The method as recited in claim 3, wherein the first time corresponds to at least five time times the time constant of the low-pass filter.

5. A method for ascertaining an internal resistance of a sensor element for acquiring a gas component from a gas mixture in a measurement gas space, the sensor element having at least one cell, the cell including at least one first electrode, at least one second electrode, and connecting solid electrolytes, an electrical voltage being capable of being measured between the first electrode and the second electrode, the method comprising the following steps:
ascertaining a reference voltage between the first electrode and the second electrode;
impressing a first current pulse having a first current by a pulse-generating unit at a first time, the first current pulse causing a charge shift in the sensor element, occurrence of the charge shift causing an increase in the electrical voltage between the first electrode and the second electrode;
ascertaining at least one voltage value between the first electrode and the second electrode at a time after an elapsing of a first settling time after the first time;
ending the first current pulse and impressing an opposite second current pulse having a second current at a second time, the opposite second current pulse causing a depolarization between the first electrode and the second electrode, and a charge shift;
ascertaining at least two voltage values between the first electrode and the second electrode at two different times after an elapsing of a second specifiable settling time after the second time;
ending the second current pulse at a third time;
ascertaining a second slope of a straight line through the at least two voltage values at the two different times;
ascertaining a voltage value at the first time as a function of the first current and of the second current and of the ascertained second slope; and
ascertaining the internal resistance of the sensor element as a function of the ascertained voltage value at the first time and the reference voltage and the first current of the first current pulse.

6. The method as recited in claim 5, wherein the specifiable first settling time and the second specifiable second settling time are determined as a function of a component property of a low-pass filter.

7. The method as recited in claim 5, wherein the sensor element is connected via a low-pass filter, the low-pass filter being connected to a control device, the low-pass filter having an associated time constant, the first time for the impressing of the first current pulse for increasing of the electrical voltage being selected such that the first time corresponds to at least three times the time constant of the low-pass filter.

8. The method as recited in claim 7, wherein the first time corresponds to at least time five times the time constant of the low-pass filter.

9. A method for ascertaining an internal resistance of a sensor element for acquiring a gas component from a gas mixture in a measurement gas space, the sensor element having at least one cell, the cell including at least one electrode, at least one second electrode, and connecting solid electrolytes, an electrical voltage being capable of being measured between the first electrode and the second electrode, the method comprising the following steps:
ascertaining at least two voltage values during a first time duration, starting at a start time and ending at a time, between the first electrode and the second electrode;
ascertaining a third slope as a function of the ascertained at least two voltage values which were ascertained during the first time duration;
impressing a first current pulse having a first current by a pulse-generating unit at a first time, the first current pulse causing a charge shift in the sensor element, occurrence of the charge shift causing an increase in the electrical voltage between the first electrode and the second electrode;
ascertaining at least one voltage value between the first electrode and the second electrode at a time after an elapsing of a first settling time after the first time;
ending the first current pulse and impressing an opposite second current pulse having a second current at a second time, the opposite second current pulse causing a depolarization between the first electrode and the second electrode, and a charge shift;
ascertaining at least two voltage values between the first electrode and the second electrode at two different times after an elapsing of a second specifiable settling time after the second time;
ending the second current pulse at a third time;
ascertaining a second slope of a straight line through the at least two voltage values at the two different times;
ascertaining a voltage value at the first time as a function of the first current and of the second current. the ascertained second slope, and a corrected slope; and
ascertaining the internal resistance of the sensor element as a function of the ascertained voltage value at the first time and the reference voltage and the first current of the first current pulse.

10. The method as recited in claim 9, wherein the first time duration is 10 ms.

11. The method as recited in claim 9, wherein the sensor element is connected via a low-pass filter, the low-pass filter being connected to a control device, the low-pass filter having an associated time constant, the first time for the impressing of the first current pulse for increasing of the electrical voltage being selected such that the first time corresponds to at least three times the time constant of the low-pass filter.

12. The method as recited in claim 11, wherein the first time corresponds to at least time five times the time constant of the low-pass filter.

13. A non-transitory electronic storage medium on which is stored a computer program for ascertaining an internal resistance of a sensor element for acquiring a gas component from a gas mixture in a measurement gas space, the sensor element having at least one cell, the cell including at least one first electrode, at least one second electrode, and connecting solid electrolytes, an electrical voltage being capable of being measured between the first electrode and the second electrode, the computer program, when executed by a computer, causing the computer to perform the following steps:
ascertaining a reference voltage between the first electrode and the second electrode;
impressing a first current pulse having a first current by a pulse-generating unit at a first time, the first current pulse causing a charge shift in the sensor element, occurrence of the charge shift causing an increase in the electrical voltage between the first electrode and the second electrode;
ascertaining between the first electrode and the second electrode at least two voltage values at two different times after an elapsing of a first specifiable settling time after the first time;
ending the first current pulse and impressing an opposite second current pulse having a second current at a second time, the opposite second current pulse causing a depolarization between the first electrode and the second electrode, and a charge shift;
ending the second current pulse at a third time;
ascertaining a linear equation as a function of the at least two voltage values and the two different times;
extrapolating a voltage value at the first time using the linear equation; and
ascertaining the internal resistance of the sensor element as a function of the extrapolated voltage value and the reference voltage and the first current of the first current pulse.

14. A control device configured to ascertain an internal resistance of a sensor element for acquiring a gas component from a gas mixture in a measurement gas space, the sensor element having at least one cell, the cell including at least one first electrode, at least one second electrode, and connecting solid electrolytes, an electrical voltage being capable of being measured between the first electrode and the second electrode, the control device being configured to:
ascertain a reference voltage between the first electrode and the second electrode;
impress a first current pulse having a first current by a pulse-generating unit at a first time, the first current pulse causing a charge shift in the sensor element, occurrence of the charge shift causing an increase in the electrical voltage between the first electrode and the second electrode;
ascertain between the first electrode and the second electrode at least two voltage values at two different times after an elapsing of a first specifiable settling time after the first time;
end the first current pulse and impress an opposite second current pulse having a second current at a second time, the opposite second current pulse causing a depolarization between the first electrode and the second electrode, and a charge shift;
end the second current pulse at a third time;
ascertain a linear equation as a function of the at least two voltage values and the two different times;
extrapolate a voltage value at the first time using the linear equation; and
ascertain the internal resistance of the sensor element as a function of the extrapolated voltage value and the reference voltage and the first current of the first current pulse.
